(19) United States
(12) Patent Application Publication
(10) Pub. No.: US 2025/0283895 A1
(43) Pub. Date: Sep. 11, 2025

(54) METHODS FOR PREDICTING AND TREATING CHRONIC LUNG ALLOGRAFT DYSFUNCTION

(71) Applicant: CHILDREN'S HOSPITAL MEDICAL CENTER, Cincinnati, OH (US)

(72) Inventors: Assem Ziady, Newport, KY (US); Don Hayes, Cincinnati, OH (US); Emily J. Skala, Cleves, OH (US); Matthew Siefert, Cincinnati, OH (US)

(21) Appl. No.: 18/859,276

(22) PCT Filed: Apr. 28, 2023
(86) PCT No.: PCT/US23/20337

§ 371 Date: Oct. 23, 2024

Related U.S. Application Data
(60) Provisional application No. 63/336,032, filed on Apr. 28, 2022.

(51) Int. Cl. G01N 33/68; A61K 31/519; A61K 39/395; C12Q 1/6883

(52) U.S. Cl. CPC G01N 33/6893; A61K 31/519; A61K 39/3955; C12Q 1/6883; C12Q 2600/118; C12Q 2600/158; G01N 2800/245; G01N 2800/50

(57) ABSTRACT

Disclosed are methods for treating a lung condition selected from one or more of lung allograft dysfunction, rejection, or failure, in an individual who has undergone a lung transplant, comprising a) detecting a biomarker; b) quantifying a biomarker level; and c) comparing the level of a biomarker to a control value; wherein a deviation in a level of biomarker indicates that said individual is likely to develop the lung condition.

### CROSS-REFERENCE TO RELATED APPLICATIONS

This application claims priority to and benefit of U.S. Application Ser. No. 63/336,032, filed Apr. 28, 2022, the contents of which are incorporated in their entirety for all purposes.

### STATEMENT REGARDING FEDERALLY-SPONSORED RESEARCH

This invention was made with government support under HL142210 awarded by the National Institutes of Health. The government has certain rights in the invention.

### BACKGROUND

CLAD, or Chronic Lung Allograft Dysfunction, is a form of allograft dysfunction in lung transplant (LTx) recipients that is the major limiting factor to long-term survival. CLAD encompasses the phenotypes of Bronchiolitis Obliterans Syndrome (BOS), Restrictive Allograft Syndrome (RAS), both BOS and RAS, or undefined. BOS is a debilitating lung condition in LTx recipients characterized by inflammation and fibrosis of the small airways, resulting in airflow obstruction and progressive lung function decline, whereas RAS is associated with alterations of the interstitium of the lung, resulting in a restrictive physiology. BOS typically develops after lung transplantation and is considered a form of chronic rejection, which occurs due to alloimmune-mediated damage to the lung tissue. CLAD, or Chronic Lung Allograft Dysfunction, encompasses a range of progressive pulmonary conditions that occur following lung transplantation, including BOS and/or RAS. CLAD is associated with poor patient outcomes and is a significant cause of morbidity and mortality in the lung transplant population. In LTx) recipients, clinical measures follow allograft outcomes (FEV1, TLC, and chest CT imaging) rather than predict them.

LTx is the only treatment option for patients with certain advanced lung diseases (e.g., cystic fibrosis (CF), pulmonary vascular disorders (PVDs), and fibrotic lung diseases), providing a chance for improved survival and quality of life. Despite a global rapid growth in LTx, the long-term outcomes have minimally improved and markedly trail behind other solid organ transplants. Despite all advancements to date, 5-year lung allograft survival is 56% for adults and 53% for children after LTx, with the primary limiting factor for long-term survival being CLAD.

Although the current diagnostic criteria for CLAD (FEV1, TLC, and chest CT imaging) in LTx recipients were recently revised with the publication of a new consensus report in 2019 by the Pulmonary Council of International Society for Heart and Lung Transplantation (ISHLT), clinicians caring for LTx recipients have been battling CLAD (bronchiolitis obliterans syndrome (BOS) or restrictive allograft syndrome (RAS) or both) since the inception of LTx. The new ISHLT CLAD phenotype classification is informative with regards to post-CLAD outcomes, but it provides little insight into predicting CLAD for LTx recipients. Prior efforts to forecast CLAD and its progression have been unsuccessful. The complicating factor in CLAD remains to be a diagnosis of exclusion with the need to exclude other etiologies affecting lung allograft function, such as ACR, antibody-mediated rejection (AMR), infection (bacterial, fungal, viral), etc.

Presently, diagnosis and intervention for chronic lung allograft dysfunction (CLAD) is driven by decline in pulmonary function, which is far less beneficial than intervening before decline manifests. Clinical interventions that would allow for prevention of CLAD development or delay of CLAD progression are lacking. The instant disclosure seeks to address one or more of the aforementioned needs in the art.

### BRIEF SUMMARY

Disclosed are methods for treating a lung condition selected from one or more of lung allograft dysfunction, rejection, or failure, in an individual who has undergone a lung transplant, comprising a) detecting a biomarker; b) quantifying a biomarker level; and c) comparing the level of a biomarker to a control value; wherein a deviation in a level of biomarker indicates that said individual is likely to develop the lung condition.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts X-rays of a 23 year old female having undergone a heart-lung transplant for bronchiolitis obliterans after previous bilateral lung transplant. The patient was admitted to ICU for acute respiratory failure unresponsive to intravenous high-dose glucocorticoids. Proteomics analysis in identified Eculizumab therapy as potential therapeutic agent. Plain radiograph showed improvement 24 hours after first Eculizumab dose as evidenced by clearance of ground glass opacity in the left lung following therapy

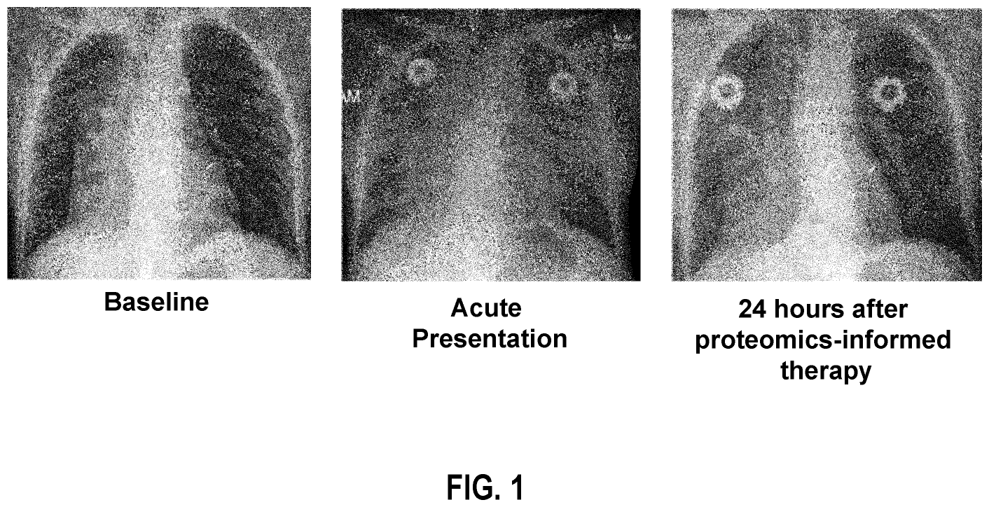

FIG. 2 depicts an HRCT scan of the chest of a 14-year-old male having undergone bilateral lung transplant for bronchiolitis obliterans after bone marrow transplant. Nov. 12, 2021 HRCT is consistent with CLAD-BOS phenotype, patient unresponsive to higher dose glucocorticoids. Proteomics analysis identified Ruxolitinib therapy as potential therapeutic agent. Following administration of Ruxolitinib, repeat HRCT of chest 3/14/22 showed stable vs minimally improved CLAD-BOS phenotype, and no progression on Ruxolitinib.

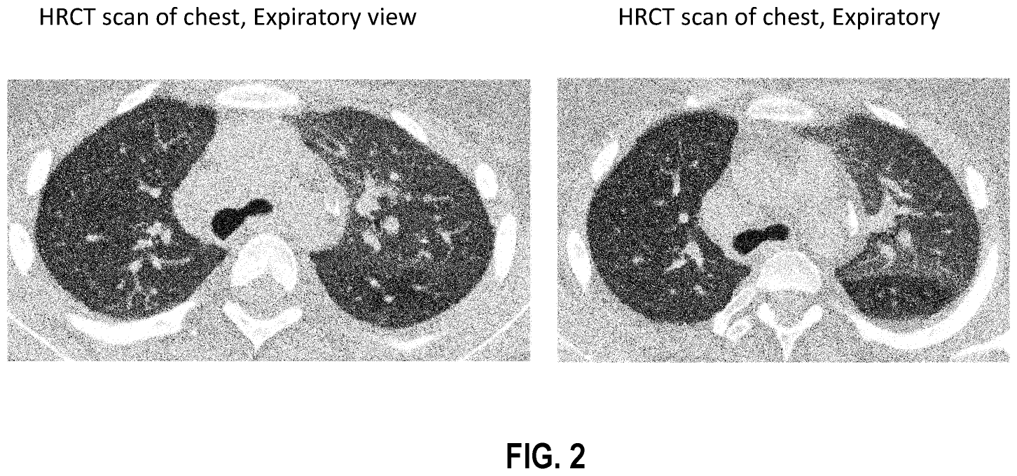

FIG. 3 is a schematic showing differential regulation of antibacterial peptide production and viral transcription in lung transplant recipients that developed BOS. Circles connote upregulation of protein expression in BAL fluid. Abbreviations: DNAH17 (Dynein Axonemal Heavy Chain 17), MAST4 (Microtubule-Associated Serine/Threonine Kinase 4), DNAH2 (Dynein Axonemal Heavy Chain 2), LRRC9 (Leucine-rich Repeat Containing 9), PCDH17 (Protocadherin 17), TANC (Tetratricopeptide Repeat, Ankyrin Repeat and Coiled-Coil Containing), GSTM1 (Glutathione S-Transferase Mu 1), GSTs (Glutathione S-Transferases), POLN (DNA Polymerase Nu). KIAA1914 is equivalent to NCBI Gene ID 168453).

FIG. 4 depicts a schematic of network analysis of differentially expressed proteins for p-value less than 0.05. Empty circles indicate a protein is upregulated in BOS; filled circles indicate down regulated in BOS. Identified proteins include IBRDC3 (IBR Domain Containing Protein 3), ERPG3 (Endoplasmic Reticulum-Golgi Intermediate Compartment Protein 3), GML (Germ Cell-Less Homolog), FANCB (Fanconi Anemia Group B Protein), BOD1L1 (Biorientation Of Chromosomes In Cell Division 1 Like 1), LRRC9 (Leucine Rich Repeat Containing 9), FAM81B (Family With Sequence Similarity 81 Member B), UAP1 (UDP-N-Acetylglucosamine Pyrophosphorylase 1), G alpha(i)-specific amine GPCRs, G alpha(q)-specific peptide GBCRs, MS4A7 (Membrane-Spanning 4-Domains, Subfamily A, Member 7), ionotropic glutamate receptor, 1,2-diacylglycerol intracellular anatomical structure, TPR (Translocated Promoter Region), TRPC (Transient Receptor Potential Canonical), NUP210 (Nucleoporin 210), NUP50 (Nucleoporin 50), ARPC4 (Actin Related Protein 2/3 Complex Subunit 4), KTN1 (Kinesin 1), RAB31P (RAB31, Member RAS Oncogene Family, Pseudogene), SLC25A42 (Solute Carrier Family 25 Member 42), DNAH17 (Dynein Axonemal Heavy Chain 17), Collagen IV (Collagen Type IV), CCDC88C (Coiled-Coil Domain Containing 88C), CATSPERB (Cation Channel Sperm Associated Beta Subunit), COBRA1 (Complex of BRCA1 and Associated Protein 1), ESM-1 (Endothelial Cell-Specific Molecule 1), MUSK (Muscle, Skeletal, Receptor Tyrosine Kinase), Rac3 (Ras-Related C3 Botulinum Toxin Substrate 3), PLC-beta (Phospholipase C-beta), PI3K reg class 1A (Phosphatidylinositol 3-Kinase Regulatory Subunit, Class IA), c-Fes (Feline Sarcoma Oncogene (v-Fes Homolog)), P13K class II, KIAA1914.

FIG. 5 depicts a schematic of network analysis of differentially expressed proteins for stringent search p-value less than 0.02; filled circles indicate down regulated in BOS. Identified proteins include ADAM-T513, MASP1, thrombin-antithrombin III, Alpha 1-antitrypsin, BRM (Brahma), Nectin-4 (Nectin Cell Adhesion Molecule 4), ADAM17 (A Disintegrin and Metalloproteinase 17), RANKL (TNFSF11) (Receptor Activator of Nuclear Factor Kappa-B Ligand (Tumor Necrosis Factor Superfamily Member 11), C1 inhibitor, semenogelin II, Tissue kallkreins, collagen IV, plasmin-alpha2 antiplasmin, desmoglein 1, BAF47 (BRG1-Associated Factor 47), ANGPTL4 (Angiopoietin-Like 4), PZP (Pregnancy Zone Protein), Furin, proinsulin, BCAN (Brevican), BAFF (TNFSF13B) (B-Cell Activating Factor (TNF Superfamily Member 13B)), BAF155 (BRG1-Associated Factor 155), A2M (Alpha-2-Macroglobulin), BMP1 (Bone Morphogenetic Protein 1), TA2MG (Thrombin-Antithrombin Complex), GATA-1 (GATA Binding Protein 1), fibrillin, asprosin, HDL proteins, LRP1 (Low-Density Lipoprotein Receptor-Related Protein 1), HSP80 (Heat Shock Protein 90).

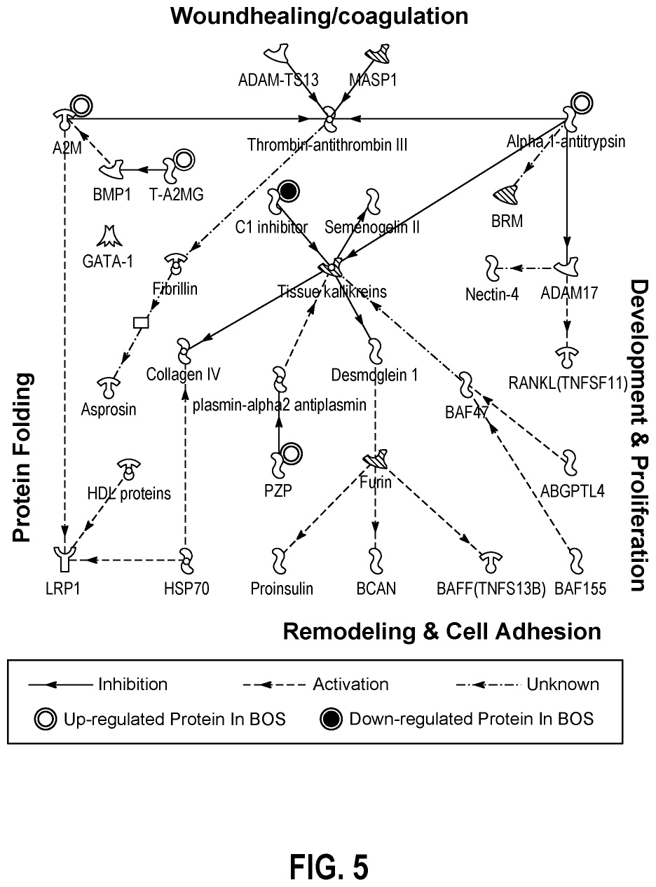

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the diagnosis, treatment, or management of a lung condition as disclosed herein.

While present diagnosis and intervention for chronic lung allograft dysfunction (CLAD) is driven by a decline in pulmonary function, such method is far less beneficial than intervening before decline manifests. As such, markers that can be used to predict allograft instability and CLAD onset may allow for established or novel clinical interventions to be implemented as a means to prevent (minimize or diminish the likelihood of) development of CLAD or to delay progression of CLAD. Markers for predicting CLAD onset are highly desirable, as they may be used to preemptively identify those at risk, allowing caregivers to tailor treatments and select interventions to prevent pulmonary decline. Disclosed herein are markers that may be used, in one aspect, for diagnosing, or otherwise identifying, individuals at risk (particularly high risk) for developing CLAD. The disclosed methods may be further used for the treatment of such individuals.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

In one aspect, a method for treating a lung condition is disclosed. The lung condition may be, for example, lung allograft dysfunction, lung allograft rejection, or lung allograft failure, in an individual who has undergone a lung transplant.

In one aspect, the method may comprise
a) detecting, in a biological sample from the individual, a biomarker selected from one or more of alpha-2 macroglobulin, tropomodulin, C1 inhibitor, PIGR (Polymeric immunoglobulin receptor), CGR (CRHR1) (Corticotropin-releasing hormone receptor 1), IRF7 (Interferon regulatory factor 7), AGP2 (ORM2) (Alpha-1-acid glycoprotein 2 orosomucoid 2), PIGR (Polymeric immunoglobulin receptor), T-A2MG (Pregnancy zone protein alpha-2-macroglobulin), C1 inhibitor (Complement C1 inhibitor), 90K (MIF-RP14) (Macrophage migration inhibitory factor-related protein 14), A2M (Alpha-2-macroglobulin), PIGR (SC) (Secretory component of the polymeric immunoglobulin receptor), Keratin 8, Keratin 19, Keratin 14, Keratin 17, Keratin 4/13, Keratin 16, actin cytoskeletal, C3b (Complement component 3b), C4 (Complement component 4), C2 (Complement component 2), antithrombin III, plasminogen, plasmin, angiotensin II, angiotensin IV, angiotensin I, angiotensinogen, C3b (Complement component 3b), iC3b (Inactivated complement component 3b), C3dg (Complement component 3dg, also known as C3d fragment), C3a (Complement component 3a), Factor I, plasmin, antithrombin III, plasminogen, C3, DNAH2 (Dynein Axonemal Heavy Chain 2), DNAH14 (Dynein Axonemal Heavy Chain 14), MAST4 (Microtubule-Associated Serine/Threonine Kinase 4), and combinations thereof;
b) quantifying a level of the one or more biomarkers detected in (a);
c) comparing said level of the one or more biomarkers to a control value;
wherein a deviation in a level of the one or more biomarkers from the control value indicates that said individual is likely to develop said lung condition.

In one aspect, the biological sample may be a bronchoalveolar lavage (BAL) fluid sample. In one aspect, the biological sample may be a blood, serum, or plasma sample from said individual. The biomarker may be a protein biomarker, or, in other aspects, may be the corresponding gene of a protein biomarker that is detected via expression of a gene that encodes for the biomarker to be detected. In one aspect, the biomarkers are detected using a targeted mass spectrometry (MS) assay.

In one aspect, the detecting of the biomarker may be carried out via enzyme-linked immunosorbent Assay (ELISA), western blotting, mass spectrometry, reverse transcription polymerase chain reaction (RT-PCR), northern blotting, in situ hybridization, microarrays, RNA sequencing (RNA-seq), Massively Parallel Signature Sequencing (MPSS), protein microarrays, or via detection of gene transcripts, such methods being known in the art.

In one aspect, the detecting may comprise detecting at least two, or at least three, or at least four, or at least five, or at least six, or at least seven, or at least eight, or at least nine, or at least 10, or at least 11, or at least 12, or at least 13, or at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, or 37 biomarkers.

The detecting may be carried out at a time point following a lung transplant, for example, one day after transplant, two days after transplant, three days after transplant, four days after transplant, five days after transplant, six days after transplant, seven days after transplant, eight days after transplant, nine days after transplant, 10 days after transplant, 11 days after transplant, 12 days after transplant, 13 days after transplant, 14 days after transplant, or greater than two weeks after transplant.

In one aspect, the control value may be a baseline value of the individual for the one or more biomarkers. The baseline value may be determined in the individual having the transplant at a time point selected from prior to transplant (e.g., within 10 years of transplant, within 5 years of transplant, within 2 years of transplant, within one year of transplant, within six months of transplant, within one month of transplant, within two weeks of transplant, within a week of transplant, the day of or the day before transplant), or immediately following transplant (i.e., within one day of transplant, within two days of transplant, within three days of transplant, within four days of transplant, within five days of transplant, within six days of transplant, within seven days of transplant, and combinations thereof). The baseline value may be an average of the levels of a biomarker obtained at various timepoints prior to transplant, or immediately following transplant. Alternatively, the control value may be a value attributed to the average of a healthy population that is representative for that patient, i.e., a similarly aged individual of same sex but without known lung dysfunction.

In one aspect, a change in a detected amount as compared to a control value (baseline or control value derived from a relevant population without lung disease) indicates that the individual is likely to develop chronic lung allograft dysfunction (CLAD). In one aspect, CLAD may be characterized by one or more of bronchiolitis obliterans syndrome (BOS) or restrictive allograft syndrome (RAS), acute cellular rejection (ACR), or combinations thereof. Where an individual is identified as likely to develop chronic lung allograft dysfunction (CLAD), the individual may be treated for CLAD.

In one aspect, the method may further comprise treating the individual with an active agent selected from one or more of a complement inhibitor (e.g. C5 inhibitor, C2 inhibitor), a JAK/STAT inhibitor, an elastase inhibitor, a hormone therapy, an anti-androgenic, an anti-coagulant, an estrogen therapy modulator, an HMG-coreductase inhibitor, an anti-parasitic, an anti-fungal, an anti-hypertensive, and anti-histamine, and anti-inflammatory, a CFTR modulator, an elastase inhibitor, a matrix remodeling inhibitor, an anti-bacterial, an anti-inflammatory, a DNAse/NET inhibitor, an antiviral, or combinations thereof, wherein said individual is diagnosed as likely to develop CLAD.

Exemplary complement inhibitor include, but are not limited to eculizumab, ravulizumab, coversin, zilucoplan, APL-2, AMY-101, and narsoplimab.

Exemplary JAK/STAT inhibitors include, but are not limited to tofacitinib, ruxolitinib, baricitinib, fedratinib, upadacitinib, itacitinib, decernotinib, PF-06651600, momelotinib, GSK2586184, pacritinib, BMS-986165, AT-9283, cerdulatinib, INCB039110, BMS-911543, LY2784544, AZD1480, SAR302503, and NS-018.

Exemplary elastase inhibitors include, but are not limited to elafin, secretory leukocyte protease inhibitor (SLPI), alpha-1 antitrypsin (AAT), SerpinA3, SerpinB1, sivelestat, AZD9668, ONO-6818, BAY-849, tosedostat, L-658,758, FICZ, PF-06741086, AZD7986, R05461111, SPK-3009, KBP-7072, ONO-5046, GW311616A, GW746027.

Exemplary hormone therapies include, but are not limited to estrogen therapy (e.g. estradiol), testosterone therapy, progesterone therapy, androgen deprivation therapy, gonadotropin-releasing hormone (GnRH) agonist therapy, GnRH antagonist therapy, aromatase inhibitor therapy, selective estrogen receptor modulator (SERM) therapy, selective estrogen receptor downregulator (SERD) therapy, thyroid hormone therapy, growth hormone therapy, adrenocorticotropic hormone (ACTH) therapy, and combinations thereof.

Exemplary anti-androgenic therapies include, but are not limited to flutamide, bicalutamide, nilutamide, enzalutamide, apalutamide, cyproterone acetate, spironolactone, ketoconazole, abiraterone acetate, degarelix, goserelin, leuprolide, histrelin, triptorelin, and combinations thereof.

Exemplary anti-coagulant include, but are not limited to heparin, warfarin, dabigatran, rivaroxaban, apixaban, edoxaban, argatroban, bivalirudin, fondaparinux, danaparoid, acenocoumarol, phenindione, nadroparin, parnaparin, certoparin, tinzaparin, dalteparin, enoxaparin, idraparinux, and drotrecogin alfa.

Exemplary estrogen therapy modulators include, but are not limited to selective estrogen receptor modulators (SERMs), selective estrogen receptor downregulators (SERDs), estrogen receptor agonists/antagonists (ERAs), estrogen receptor beta agonists (EROAs), tissue selective estrogen complexes (TSECs), and estetrol (E4).

Exemplary HMG-coreductase inhibitors include, but are not limited to atorvastatin, fluvastatin, lovastatin, pitavastatin, pravastatin, rosuvastatin, and simvastatin.

Exemplary anti-parasitics include, but are not limited to chloroquine, hydroxychloroquine, ivermectin, metronidazole, nitazoxanide, praziquantel, pyrantel, quinine, albendazole, mebendazole, artemisinin, artemether-lumefantrine, pyrimethamine, trimethoprim-sulfamethoxazole, and combinations thereof.

Exemplary anti-fungals include, but are not limited to amphotericin B, fluconazole, itraconazole, ketoconazole, caspofungin, micafungin, voriconazole, terbinafine, griseofulvin, and nystatin.

Exemplary anti-hypertensives include, but are not limited to diuretics (e.g. hydrochlorothiazide, furosemide), ACE inhibitors (e.g. lisinopril, enalapril), angiotensin II receptor blockers (ARBs) (e.g. losartan, valsartan), beta blockers (e.g. metoprolol, atenolol), calcium channel blockers (e.g. amlodipine, verapamil), alpha blockers (e.g. doxazosin, prazosin), central agonists (e.g. clonidine, methyldopa), renin inhibitors (e.g. aliskiren), and vasodilators (e.g. hydralazine, minoxidil).

Exemplary anti-histamine include, but are not limited to diphenhydramine, chlorpheniramine, loratadine, cetirizine, fexofenadine, desloratadine, cetirizine/pseudoephedrine, loratadine/pseudoephedrine, and combinations thereof.

Exemplary anti-inflammatories include, but are not limited to nonsteroidal anti-inflammatory (NSAIDs), COX-2 inhibitors, corticosteroids, immunomodulators, and biologics (e.g. infliximab, adalimumab).

Exemplary CFTR modulators include, but are not limited to ivacaftor, lumacaftor/ivacaftor, tezacaftor/ivacaftor, elexacaftor/tezacaftor/ivacaftor, and combinations thereof.

Exemplary elastase inhibitors include, but are not limited to, elafin, secretory leukocyte protease inhibitor (SLPI), alpha-1-antitrypsin, SerpinA1, SerpinB1, SerpinB3, SerpinB4, SerpinB6, SerpinE1, SerpinF2, and combinations thereof.

Exemplary matrix remodeling inhibitors include, but are not limited to collagenase inhibitors, elastase inhibitors, matrix metalloproteinase (MMP) inhibitors, tissue inhibitors of metalloproteinases (TIMPs), plasminogen activator inhibitors (PAIs), hyaluronidase inhibitors, and combinations thereof.

Exemplary anti-bacterials include, but are not limited to, penicillins, cephalosporins, carbapenems, monobactams, tetracyclines, macrolides, aminoglycosides, fluoroquinolones, sulfonamides, nitrofurans, metronidazole, vancomycin, linezolid, daptomycin, polymyxins, fosfomycin, and combinations thereof.

Exemplary DNAse/NET inhibitors include, but are not limited to, Alpha-1 antitrypsin (AAT), DNase I, Recombinant human deoxyribonuclease (rhDNase), Dornase alfa, Elafin, Secretory leukocyte protease inhibitor (SLPI), al-antichymotrypsin, al-macroglobulin, and combinations thereof.

Exemplary antivirals include, but are not limited to acyclovir, amantadine, atazanavir, cidofovir, darunavir, efavirenz, enfuvirtide, famciclovir, foscarnet, ganciclovir, indinavir, lopinavir, maraviroc, nevirapine, oseltamivir, ribavirin, ritonavir, saquinavir, sofosbuvir, telaprevir, tenofovir, valaciclovir, zanamivir, and combinations thereof.

In one aspect, the treatment of the individual likely to develop CLAD may include administration of one or more of eculizumab, ruxolitinib, estradiol, abiraterone, acenocoumarol, afimoxifene, apomine, amodiaquine, amophotericin B, cyclothiazide, cetirizine, cortisone, curcumin, alvelestat, batismatat, doxycycline, azithromycin, pulmozyme, interferon, and combination thereof.

In one aspect, the individual may have one or more conditions selected from cystic fibrosis (CF), pulmonary vascular disorders (PVD), idiopathic pulmonary fibrosis, chronic obstructive pulmonary disease (COPD), alpha-1 antitrypsin deficiency, or combinations thereof. In certain aspects, the individual may be a pediatric patient, or an adult patient.

In a further aspect, disclosed is a method for treating a lung condition selected from one or more of lung allograft dysfunction, rejection, or failure, in an individual who has undergone a lung transplant, which may comprise assaying for a condition in the individual selected from a skin and connective tissue disease, stromatognathic disease, mouth disease, autoimmune disease, connective tissue disease, rheumatoid arthritis, rheumatic diseases, arthritis, joint disease, sprain/strain, wound/injury, erythema multiforme, and combinations thereof, wherein when the condition is present in the individual, the individual is treated as having a higher likelihood of developing CLAD as set forth above. The detection of a biomarker as described above may be combined with the detection of the one or more conditions, wherein both the biomarker and presence of one or more conditions may be used to identify an individual as likely to develop CLAD, who may be further administered a treatment as described herein.

In a further aspect, disclosed is a method for treating lung allograft dysfunction, rejection, or failure in an individual who has undergone a lung transplant, which may comprise assaying for a status in in individual selected from platelet degranulation, regulation of complement activation (lectin pathway), negative regulation of complement activation (lectin pathway), negative regulation of blood coagulation (intrinsic pathway), regulated exocytosis, regulation of blood coagulation (intrinsic pathway) exocytosis, negative regulation of protein activation cascade, regulation of protein activation cascade, secretion by cell, cilium-dependent cell motility, cilium or flagellum-dependent cell motility, response to fungicide, determination of left/right asymmetry in nervous system, regulation of glutamate metabolic process, ionotropic glutamate receptor signaling pathway, protein localization to motile cilium, cilium movement, regulation of glutamine family amino acid metabolic process, regulation of NMDA receptor activity, or combinations thereof, wherein when a change in status is detected, the individual is treated as having a higher likelihood of developing CLAD. The detection of one or more biomarkers as described above, and/or the detection of one or more conditions as described above, may be combined with the detection of the one or more statuses, wherein both the biomarker and presence of one or more conditions may be used to identify an individual as likely to develop CLAD, who may be further administered a treatment as described herein.

In a further aspect, disclosed is a method for treating lung allograft dysfunction, rejection, or failure in an individual who has undergone a lung transplant, which may comprise assaying for a component in the antibacterial peptide production pathway (e.g. testing for Kallikrein pathway components, kallikrein 1-15), a viral transcription pathway (e.g. glucocorticoid regulation of antiviral responses), a cytoskeleton remodeling pathway, a lectin induced complement pathway, a blood coagulation pathway, an angiotensin system maturation pathway, a viral-associated coagulopathy pathway, a complement pathway, a plasminogen activator pathway, and combinations thereof. The detection of one or more biomarkers as described above, and/or the detection of one or more conditions as described above, and/or assaying for one or more statuses as described above, may be combined with the detection of the one or more statuses, wherein both the biomarker and presence of one or more conditions may be used to identify an individual as likely to develop CLAD, who may be further administered a treatment as described herein.

In one aspect, a plurality of detection agents specific for two or more biomarkers are disclosed herein. Such detections agents may include antibodies capable of detecting one or more antibodies, or oligonucleotides specific for RNA that encodes for one or more biomarker as disclosed herein. Further provided are kits for carrying out the methods, which may include one or more of an ELISA plate pre-coated with antibodies specific for one or more biomarkers, sample collection containers and reagents for sample preparation or preservation, RNA extraction reagents, PCR reagents including primers specific for one or more biomarkers, and a control reagent (positive and negative control reagent).

In one aspect, a method of treating chronic lung allograft dysfunction (CLAD) in an individual in need thereof is disclosed, the method comprising administering an effective amount of eculizumab to said individual.

In one aspect, a method of treating chronic lung allograft dysfunction (CLAD) in an individual in need thereof is disclosed, the method comprising administering an effective amount of ruxolitinib to said individual.

In one aspect, a method of treating chronic lung allograft dysfunction (CLAD) in an individual in need thereof is disclosed, the method administering an effective amount of an agent selected from estradiol, abiraterone, acenocoumarol, afimoxifene, apomine, amodiaquine, amophotericin B, cyclothiazide, cetirizine, cortisone, curcumin, alvelestat, batismatat, doxycycline, azithromycin, pulmozyme, interferon, or combination thereof to said individual.

The administration may be carried out for a period of time and at a dosage sufficient to achieve the desired result, i.e., prevention of, delayed progression of, or resolution of CLAD in the individual.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Applicant identified proteome biomarkers in BAL fluid segregating CLAD/BOS from no CLAD/BOS after onset of disease. BAL fluid samples collected from 31 adult LTx recipients with and without CLAD/BOS (Table 1) were analyzed, using protein-adsorption columns and gel and column chromatography. A total of 4,908 protein isoforms were present in at least 50% of either cohort, with 227 isoforms (p<0.01, FDR<0.1) distinguishing LTx recipients with and without CLAD/BOS. Data were normalized to a relative abundance (RA) measure (0 to 1) for each sample. For each isoform, RA was summarized (mean RA, number with RA>0, ratio of RA control/BOS) and, to reduce the data, a battery of paired statistical tests were performed on the matched samples: McNemar's, Wilcoxon Signed-Rank, paired Student's t test, and permutation of the difference.

TABLE 1
Demographic and characteristics of study cohort.

| | | |
|---|---|---|
| Variable | BOS (N = 15) | No BOS (N = 16) |

| | | |
|---|---|---|
| Age (years) | 66.4 ± 8.3 | 63.8 ± 5.4 |

| | | | | |
|---|---|---|---|---|
| Sex (Female, %) | 2 | (13%) | 3 | (19%) |
| Indication for LTx | | | | |
| COPD (%) | 2 | (13) | 6 | (38%) |
| IPF (%) | 12 | (80%) | 7 | (44%) |
| A1AD (%) | 1 | (7%) | 1 | (6%) |
| HP (%) | | | 1 | (6%) |
| Mixed CTD-ILD (%) | | | 1 | (6%) |

| | | |
|---|---|---|
| FVC (Liters) | 3.07 ± 1.12 | 2.98 ± 0.87 |
| FVC (% predicted) | 67.4 ± 19.7 | 70.9 ± 17.2 |
| FEV1 (Liters) | 1.95 ± 0.66 | 2.44 ± 0.76 |
| FEV1 (% predicted) | 56.1 ± 19.1 | 74.6 ± 21.3 |

| | | |
|---|---|---|
| BOS Grade | | |
| Grade 0p | 3 | (20%) |
| Grade 1 | 8 | (53%) |
| Grade 2 | 3 | (20%) |
| Grade 3 | 1 | (7%) |

BOS = bronchiolitis obliterans syndrome, LTx = lung transplant, COPD = chronic obstructive pulmonary disease; IPF = idiopathic pulmonary fibrosis; A1AD = alpha-1 antitrypsin deficiency; HP = hypersensitivity pneumonitis; CTD-ILD = connective tissue disease-interstitial lung disease; FVC = forced vital capacity; FEV1 = forced expiratory volume in one second.

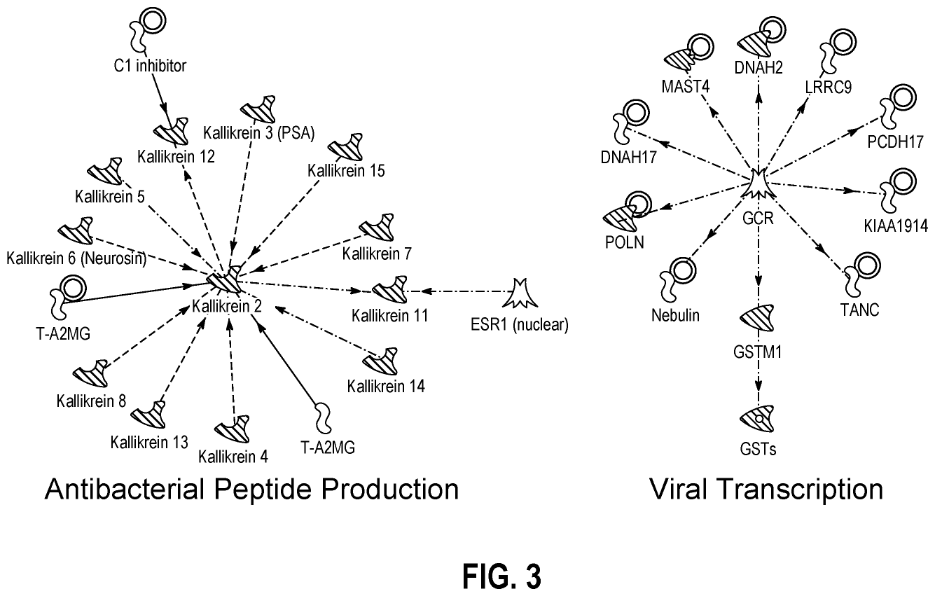

Dimensionality reduction methods were applied including principal component analysis (PCA), logistic regression with LASSO and random forests to reduce the dataset from the almost 5,000 isoforms analyzed to 227 isoforms with p<0.01, FDR<0.1 across the battery of tests. Pathway analysis of differentially expressed proteins reveal vascular permeability (p=3.31×10⁻³), proliferation/migration (p=5.68×10⁻²), protein localization to ciliary membrane (p=8.02×10⁻⁴), and prostaglandin E2 (PGE2) immune response (p=4.93×10⁻²). A more stringent percolator-decoy analysis identified 9 isoform differences and reveals changes in platelet degranulation (p=5.36×10⁻⁸, FDR=6.09×10⁻⁶), kallikrein-kinin driven inflammation (p=3.37×10⁻⁴, FDR=3.37×10⁻³), lectin-induced complement pathway (p=6.19×10⁻⁸, FDR=6.09×10⁻⁶), blood coagulation (p=6.19×10⁻⁷, FDR=4.57×10⁻⁵), and IL-6 mediated inflammation (p=3.98×10⁻³, FDR=9.96×10⁻³). Strong signals for antigen presentation and immunoglobulin mediated immune response (p=9.76×10⁻¹⁷) and viral transcription (p=6.41×10⁻¹¹) were also observed in the BOS cohort. The data suggest that antiviral/antimicrobial, complement activation, and antigen presentation are features of disease in LTx patients that developed CLAD/BOS. FIG. 3 shows response pathways to viral and bacterial infection that are implicated in the data as highly correlated with CLAD/BOS.

Further bioinformatic analysis of top pathways identified 1182 protein isoforms that were present in at least 50% of either cohort, with 298 isoforms (p<0.05, FDR<0.1, Table 2A) or 16 isoforms (p<0.2, FDR<0.01, Table 2B) distinguishing LTx recipients with and without CLAD/BOS. For stringent FDR cutoff (<0.01), pathway analysis of differences indicated alterations in antimicrobial peptide production (p=4.90×10⁻¹²), platelet degranulation (p=6.37×10⁻⁸), regulation of complement activation by lectin (p=6.75×10⁻⁸), negative regulation of blood coagulation (p=6.75×10⁻⁷), and IL-6 mediated inflammation (p=3.93×10⁻³). For moderate FDR cutoff (<0.1), chief distinguishing features include viral gene expression (p=2.48×10⁻²¹), glutathione metabolism (p=2.20×10⁻¹⁵), and membrane protein targeting (p=3.05×10⁻¹⁴). Disease associations of identified differences included connective tissue disorders (p=2.15×10⁻⁵) and wounds and injuries (p=3.30×10⁻⁵). Analyses of marker association with disease revealed strong associations with autoimmune diseases, highlighting the involvement of immunity in CLAD/BOS (Table 3). The associations are biological validation that the markers point to features of disease that are known to occur in CLAD/BOS.

TABLE 2
GO Process (Gene Ontology) analysis for differentially expressed proteins:
(A) p-value <0.05, (B) stringent search p-value <0.2.

| | | |
|---|---|---|
| Processes | p-value | FDR |

A

| | | |
|---|---|---|
| negative regulation of complement activation, lectin pathway | 5 296E−05 | 2.504E−03 |
| regulation of complement activation, lectin pathway | 5.296E−05 | 2.504E−03 |
| positive regulation of single stranded viral RNA replication via | 1.581E−04 | 5.292E−03 |
| double stranded DNA intermediate | | |
| negative regulation of blood coagulation, intrinsic pathway | 5.220E−04 | 1.219E−02 |
| regulation of glutamate metabolic process | 7.792E−04 | 1.541E−02 |
| establishment of protein localization to plasma membrane | 1.169E−03 | 1.933E−02 |
| regulation of pulmonary blood vessel remodeling | 7.296E−03 | 5.809E−02 |
| negative regulation of complement activation | 7.503E−03 | 5.936E−02 |
| regulation of response to reactive oxygen species | 7.593E−03 | 5.956E−02 |
| proteolysis involved in cellular protein catabolic process | 7.606E−03 | 5.956E−02 |

B

| | | |
|---|---|---|
| platelet degranulation | 5.299E−10 | 2.062E−07 |
| acute-phase response | 7.325E−09 | 1.425E−06 |
| regulated exocytosis | 5.622E−08 | 6.255E−06 |
| regulation of complement activation, lectin pathway | 8.258E−08 | 6.255E−06 |
| negative regulation of blood coagulation, intrinsic pathway | 8.254E−07 | 2.532E−05 |
| acute inflammatory response to antigenic stimulus | 5.1765−05 | 5.298E−04 |
| inflammatory response | 7.674E−05 | 7.281E−04 |
| inflammatory response to antigenic stimulus | 2.709E−04 | 2.228E−03 |
| chronic inflammatory response to antigenic stimulus | 2.149E−03 | 9.951E−03 |
| lymphocyte mediated immunity | 1.178E−02 | 3.580E−02 |

TABLE 3
Disease associations for differentially expressed proteins. (A)
p-value <0.05, (B) stringent search p-value <0.2.

| | | |
|---|---|---|
| Conditions | p-value | FDR |

A

| | | |
|---|---|---|
| Skin and Connective Tissue Diseases | 1.269E−17 | 1.035E−15 |
| Stomatognathic Diseases | 9.415E−12 | 2.318E−10 |
| Mouth Diseases | 3.747E−11 | 8.767E−10 |
| Autoimmune Diseases | 9.011E−05 | 7.396E−04 |
| Connective Tissue Diseases | 2.607E−02 | 8.724E−02 |

B

| | | |
|---|---|---|
| Arthritis, Rheumatoid | 2.440E−07 | 3.237E−05 |
| Rheumatic Diseases | 9.549E−07 | 7.396E−05 |
| Arthritis | 9.968E−07 | 7.396E−05 |
| Joint Diseases | 1.167E−06 | 7.396E−05 |
| Sprains and Strains | 1.301E−06 | 7.396E−05 |
| Wounds and Injuries | 3.145E−06 | 1.252E−04 |
| Connective Tissue Diseases | 3.674E−06 | 1.329E−04 |
| Mouth Diseases | 1.480E−04 | 2.561E−03 |
| Erythema Multiforme | 1.960E−04 | 3.120E−03 |
| Stomatognathic Diseases | 6.520E−04 | 5.767E−03 |

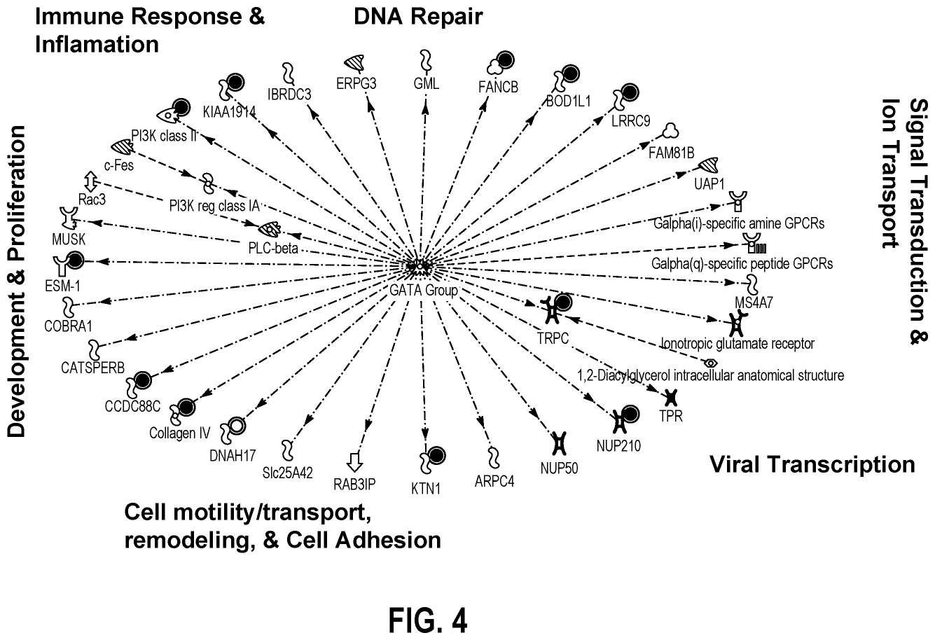

Additional highly significant networks connected immunity to viral transcription, cell proliferation, tissue remodeling, and DNA damage (FIG. 4A). Network analysis also connected WNT wound healing pathways to cell adhesion and remodeling, and protein misfolding and ER stress (FIG. 4B).

Proteomic N of 1 studies of blood and BAL fluid in patients suspected of developing CLAD: Applicant further developed an analysis approach to examine BAL fluid and blood samples from an individual patient and delineate changes in signaling pathways that contribute to the clinical presentation of disease following lung transplant. In these N of 1 studies, strong statistical rigor was achieved, generating bioinformatics data with highly significant findings. For example, for a patient with advancing CLAD following LTx, analyses of both BAL fluid and blood revealed increased lectin-induced complement signaling, blood coagulation, and viral transcription (Table 4). The BAL fluid and blood results were very concordant, with the BAL fluid data achieving more significant identification of dysregulation in lectin-induced complement signaling, blood coagulation, and viral transcription than the blood data (p˜1×10⁻²³-1×10⁻⁹ for BALF vs. p˜1×10⁻¹³-1×10⁻⁷ for blood, Table 4).

TABLE 4
N of 1 analysis of patient with progressive CLAD. (A) Processes and associated
markers in blood, (B) Processes and associated markers in BAL.

| Disease Processes | P-Value | FDR | Markers |
|---|---|---|---|
| Cytoskeleton remodeling | 1.60E−26 | 5.79E−24 | Keratin 8, Keratin 19, |
| | | | Keratin 14, Keratin 17 |
| | | | Keratin 4/13, Keratin 16 |
| | | | Actin cytoskeletal |
| Lectin induced complement pathway | 1.67E−23 | 3.01E−21 | C3b, C4, C2, C1 inhibitor |
| Blood coagulation | 1.15E−09 | 5.20E−08 | Antithrombin III, |
| | | | Plasminogen, Plasmin |
| Angiotensin system maturation | 6.63E−09 | 2.66E−07 | Angiotensin II, Angiotensin |
| | | | IV, Angiotensin I, |
| | | | Angiotensinogen, |
| Viral-associated coagulopathy | 1.09E−08 | 3.59E−07 | C3b, C4, C3a |
| Lectin induced complement pathway | 1.16E−13 | 3.62E−12 | C3c, C3, C3b, iC3b, C3dg, |
| | | | C3a, Factor I |
| Blood coagulation | 1.42E−07 | 1.44E−06 | Plasmin, Antithrombin III, |
| | | | Plasminogen |
| Complement pathway disruption in thrombotic | 1.42E−07 | 1.44E−06 | C3, C3b, C3a, Factor I |
| microangiopathy | | | |
| Plasminogen activators signaling | 1.09E−05 | 9.49E−05 | Plasmin, Plasminogen |
| Viral-associated coagulopathy | 3.42E−05 | 2.61E−04 | C3, C3b, C3a |

Studies in adult cohort comparisons and N of 1 studies (both above) highlighted a number of signaling cascades as chief markers of development of CLAD. Lectin-induced complement activation, blood coagulation, and viral transcription can be linked to the clinical features of disease, and all strongly associated with CLAD and graft rejection. Markers for complement activation and viral transcription were significantly elevated in CLAD, while markers for blood coagulation were significantly decreased in CLAD. Proteomic analysis employs mass spectrometry-based approaches for the identification/quantitation of proteins in serum, plasma, urine, and tissue samples. (See, e.g., Ziady A G, Kinter M. Protein sequencing with tandem mass spectrometry. Methods Mol. Biol. 2009; 544:325-41.) For plasma, whole protein may be prepared using albumin adsorption affinity columns or beads. For BAL fluid, no albumin depletion is necessary. Gel and column chromatography may be used to fractionate samples to increase the number of proteins that can be analyzed and improve coverage (amount of sequence identified per protein). Following fractionation, samples may be subjected to tryptic digestion prior to detection via MS, as previously described. Briefly, samples are loaded in a HPLC system autosampler and eluted by reverse-phase chromatography into an LTQ velos-pro mass spectrometer fitted with a nanospray ion source for highly sensitive detection and analysis.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method for treating a lung condition selected from one or more of lung allograft dysfunction, rejection, or failure, in an individual who has undergone a lung transplant, comprising
a) detecting, in a biological sample from said individual, one or more biomarkers selected from one or more of alpha-2 macroglobulin, tropomodulin, C1 inhibitor, PIGR (Polymeric immunoglobulin receptor), CGR (CRHR1) (Corticotropin-releasing hormone receptor 1), IRF7 (Interferon regulatory factor 7), AGP2 (ORM2) (Alpha-1-acid glycoprotein 2 orosomucoid 2), PIGR (Polymeric immunoglobulin receptor), T-A2MG (Pregnancy zone protein alpha-2-macroglobulin), C1 inhibitor (Complement C1 inhibitor), 90K (MIF-RP14) (Macrophage migration inhibitory factor-related protein 14), A2M (Alpha-2-macroglobulin), PIGR (SC) (Secretory component of the polymeric immunoglobulin receptor), Keratin 8, Keratin 19, Keratin 14, Keratin 17, Keratin 4/13, Keratin 16, actin cytoskeletal, C3b (Complement component 3b), C4 (Complement component 4), C2 (Complement component 2), antithrombin III, plasminogen, plasmin, angiotensin II, angiotensin IV, angiotensin I, angiotensinogen, C3b (Complement component 3b), iC3b (Inactivated complement component 3b), C3dg (Complement component 3dg, also known as C3d fragment), C3a (Complement component 3a), Factor I, plasmin, antithrombin III, plasminogen, C3, DNAH2 (Dynein Axonemal Heavy Chain 2), DNAH14 (Dynein Axonemal Heavy Chain 14), MAST4 (Microtubule-Associated Serine/Threonine Kinase 4), and isoforms and combinations thereof;
b) quantifying a level of said one or more biomarkers detected in (a); and
c) comparing said level of said one or more biomarkers to a control value;
wherein a deviation in a level of said one or more biomarkers from said control value indicates that said individual is likely to develop said lung condition.

2. The method of claim 1 wherein said biological sample is a bronchoalveolar lavage (BAL) fluid sample.

3. The method of claim 1, wherein said biological sample is a blood sample.

4. The method of claim 1, wherein said biomarker is a protein biomarker.

5. The method of claim 1, wherein said biomarker is detected via expression of a gene that encodes for said biomarker.

6. The method of claim 1, wherein said detecting comprises detecting at least two, or at least three, or at least four, or at least five, or at least six, or at least seven, or at least eight, or at least nine, or at least 10, or at least 11, or at least 12, or at least 13, or at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, or 37 biomarkers.

7. The method of claim 1, wherein said detecting of said biomarker occurs at a time point selected from one day after transplant, two days after transplant, three days after transplant, four days after transplant, five days after transplant, six days after transplant, seven days after transplant, eight days after transplant, nine days after transplant, 10 days after transplant, 11 days after transplant, 12 days after transplant, 13 days after transplant, 14 days after transplant, or greater than two weeks after transplant.

8. The method of claim 1, wherein said control value is a level of said one or more biomarkers as determined at a time point selected from prior to transplant, the day of transplant, within one day of transplant, within two days of transplant, within three days of transplant, within four days of transplant, within five days of transplant, within six days of transplant, within seven days of transplant, and combinations thereof.

9. The method of claim 1, wherein said detection is carried out via ELISA, mass spectrometry proteomics, or a combination thereof.

10. The method of claim 8, wherein a change in a detected amount as compared to a control value indicates that said individual is likely to develop chronic lung allograft dysfunction (CLAD), and said individual is treated for CLAD.

11. The method of claim 10, wherein said CLAD is characterized by one or more of bronchiolitis obliterans syndrome (BOS) or restrictive allograft syndrome (RAS), acute cellular rejection (ACR), or combinations thereof.

12. The method of claim 1, further comprising treating said individual with an active agent selected from one or more of a complement inhibitor, a JAK/STAT inhibitor, an elastase inhibitor, a hormone therapy, an anti-androgenic, an anti-coagulant, an estrogen therapy modulator, an HMG-coreductase inhibitor, an anti-parasitic, an anti-fungal, an anti-hypertensive, and anti-histamine, and anti-inflammatory, a CFTR modulator, an elastase inhibitor, a matrix remodeling inhibitor, an anti-bacterial, an anti-inflammatory, a DNAse/NET inhibitor, an antiviral, or combinations thereof, wherein said individual is diagnosed as likely to develop CLAD.

13-31. (canceled)

32. The method of claim 10, comprising treating said individual likely to develop CLAD with an active agent selected from one or more of eculizumab, ruxolitinib, estradiol, abiraterone, acenocoumarol, afimoxifene, apomine, amodiaquine, amophotericin B, cyclothiazide, cetirizine, cortisone, curcumin, alvelestat, batismatat, doxycycline, azithromycin, pulmozyme, interferon, or combination thereof.

33. The method of claim 1, wherein said individual is diagnosed with cystic fibrosis (CF), pulmonary vascular disorders (PVD), idiopathic pulmonary fibrosis, chronic obstructive pulmonary disease (COPD), alpha-1 antitrypsin deficiency, or combinations thereof.

34. The method of claim 1, wherein said individual is a pediatric patient.

35. The method of claim 1, wherein said individual is an adult patient.

36. A method for treating a lung condition, said lung condition selected from one or more of lung allograft dysfunction, rejection, or failure, in an individual who has undergone a lung transplant, comprising assaying for a condition in said individual selected from a skin and connective tissue disease, stromatognathic disease, mouth disease, autoimmune disease, connective tissue disease, rheumatoid arthritis, rheumatic diseases, arthritis, joint disease, sprain/strain, wound/injury, erythema multiforme, and combinations thereof, wherein when said condition is present, said individual is treated as having a higher likelihood of developing CLAD.

37. (canceled)

38. (canceled)

39. A method of treating chronic lung allograft dysfunction (CLAD) in an individual in need thereof, comprising administering an effective amount of one or both of eculizumab and ruxolitinib to said individual.

40. (canceled)

41. (canceled)

42. The method of claim 39, wherein eculizumab is administered.

43. The method of claim 39, wherein ruxolitinib is administered.
